(19) United States
(12) Patent Application Publication
(10) Pub. No.: US 2008/0228039 A1
(43) Pub. Date: Sep. 18, 2008

(54) Semi-passive method and system for monitoring and determining the status of an unattended person

(75) Inventors: Steve D. Huseth, Plymouth, MN (US); Liana M. Kiff, Minneapolis, MN (US)

Correspondence Address: Kris T. Fredrick;Honeywell International Inc., 101 Columbia Rd., P.O. Box 2245, Morristown, NJ, 07962, US

(73) Assignee: Honeywell International Inc.

(21) Appl. No.: 11/717,570

(22) Filed: Mar. 12, 2007

(51) Int. Cl. A61B 5/00; G08B 23/00

(52) U.S. Cl. 600/300; 340/573.1

(57) ABSTRACT

A method and system for determining the status and monitoring an unattended individual. A radio beacon device can be associated with one or more individuals (e.g., an unattended person). Behavioral information about the individual(s) can be collected by tracking the location of the individual within a particular area (e.g., home, assisted living facility, etc.) utilizing the radio beacon device. The presence of other persons can then be distinguished from that of the individual within said particular area utilizing said behavioral information collected about said individual, thereby permitting a determination of the status of said individual and distinguishing the presence of other persons within said particular area. The behavioral information can include, for example, one or more activity levels associated with the individual.

### TECHNICAL FIELD

Embodiments are generally related to data-processing methods and systems. Embodiments are additionally related to wireless communication methods and systems location tracking technology. Embodiments are also related to technologies for use in healthcare and long-term care facilities.

### BACKGROUND OF THE INVENTION

Many people, at some point in their lives, have to cope with the burden of arranging for the care of an aging parent or loved one. When an elderly person begins to exhibit signs that he or she is unable to care for themselves safely, often the child must start thinking about whether their parent or loved one will require some type of assistance, and what form that assistance should take. In the beginning, it may be sufficient to arrange for an assistant to visit the elder's home on a periodic basis. As the elderly person's condition worsens, however, he or she may eventually have to be placed in a nursing facility or some other form of assisted living facility.

Family members may make precipitous decisions about care to address their own peace-of-mind, in the absence of real information about their loved-one's day-to-day quality of life. Quality of care, whether the care is delivered to the home, or administered at a living facility, is a constant worry for the remote caregiver. Nursing facilities of all types are plagued with high turnover and a lack of skilled workers to fill nursing and aide positions. The quality of care in many facilities consequently suffers.

The range of services available at every level of care is widely different, and small changes in needs often require momentous changes in living arrangements. For example, someone who begins to need assistance with eating or bathing may be forced to move from an independent living apartment to an assisted living apartment, due to regulations in the level of care that each type of facility is authorized to provide. Each of these moves adds further stress to the frail individual, and can precipitate a rapid decline in his or her condition.

Location tracking technology has been utilized for monitoring individuals where the location of a mobile beacon is detectable by an array of anchor receivers. A beacon located on an object or person transmits a radio signal that is received by the array of anchors. Since a radio signal attenuates at a known rate over distance, measuring the strength of the signal at the receiver allows the receiver to calculate an estimated distance to the mobile device. Combining the distance measurements from an array of anchor receivers placed in known locations using a triangulation algorithm, allows the location of the beacon can be determined. In most home situations, however, it is not cost effective to precisely place each of the anchors at a particular location. Additionally, such location tracking technology alone does not provide for behavioral information about the individual being tracked. Such technology also does not effectively distinguish one person from another within a particular location, such as a home environment.

Accordingly, a need exists for an improved method and system for accurately and efficiently determining the status and monitoring the location of an individual, such as an elderly or handicapped person.

### BRIEF SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the present invention to provide for an improved data-processing method and system.

It is another aspect of the present invention to provide for a method and system for monitoring the frequency and duration of the care of an individual, such as a handicapped and/or elderly person.

It is an additional aspect of the present invention to provide for a method and system for facilitating the care of an individual, such as a handicapped and/or elderly person, residing primarily in a long-term care facility and/or a room and/or home care environment or any other healthcare facility.

It is another aspect of the present invention to provide for a location tracking method and system for monitoring an unattended person.

It is a further aspect of the present invention to provide for a semi-passive method and system to determine the location and status of an unattended person.

It is a further aspect of the present invention to provide the ability to disambiguate data from other actors in the environment, such that these signals do not interfere with an accurate assessment of the monitored patient.

It is a further aspect of the present invention to provide the ability to track both human and/or non-human (e.g., domestic pets) inhabitants of a particular space.

The aforementioned aspects and other objectives and advantages can now be achieved as described herein. A method and system for determining the status and monitoring of an unattended individual are disclosed. In general, a radio beacon device is associated with an individual (e.g., an unattended person). Behavioral information about the individual can then be collected by tracking the location of the individual within a particular area (e.g., a home) utilizing the radio beacon device. The presence of other persons can then be distinguished from that of the individual within the particular area utilizing the behavioral information collected about the individual, thereby permitting a determination of the status of the individual and distinguishing the presence of other persons within the particular area. The behavioral information can include, for example, one or more activity levels associated with the individual.

Additionally, an array of motion detection sensors can be placed within one or more selected locations of the particular area, wherein the array of motion detection sensors generates motion sensor data. The motion sensor data generated by the array of motion detection sensors and the behavioral information associated with the individual can be utilized to determine if the motion sensor data and the behavioral information agree or differ. If the motion sensor data and the behavioral information agree with each other, an assumption can be generated indicating that the individual is alone within the particular area. If, however, the motion sensor data and the behavioral information do not agree with each other, an assumption is generated indicating that more than one individual is present within the particular area. A triangulation algorithm can also be utilized for determining the location of the radio beacon device located with the individual.

The present invention can thus utilize two separate location tracking methods to collect behavioral information on the client and distinguish the presence of other persons on the home; one being a radio beacon device carried by the client, and second being an array of motion detection sensors placed in selected rooms or zones of the home. Both systems are capable of determining the room the client is in. When the client is alone, both systems will generally show the person in the same room or zone of the home. However, when another person is in the home, the room identified by the two tracking systems will tend to be different. Furthermore, using a radio beacon device, the behavior of the client can be distinguished from the actions of a second person in the home providing more accurate behavioral data about the client. Finally, if the client stops wearing the beacon device, the motion data can still be used as a backup.

Conditions such as Alzheimer's disease, other forms of dementia, and other forms of cognitive disability (e.g., autism) can create a situation where the patient can not or will not wear a tracking device on their person. In this case, the aforementioned approaches may be utilized to monitor the individuals, domestic pets, and environment around the patient and extract patient behavior data necessary to deliver appropriate care. For example, it is known that cognitively disabled patients are prone to wander. Patients cared for at home routinely “escape” their caregivers and, too frequently, are exposed to the elements, or may suffer abuse at the hands of strangers. These same patients are likely to remove the tracking devices that could prevent such events.

The system described herein may thus be adapted for use in monitoring the behavior of caregivers such that the normal behaviors of able members of an environment may be ignored, and dangerous behaviors of unmonitored individuals can be immediately recognized. For example, smart door locks may automatically permit monitored caregivers to leave while preventing the exit of un-tracked, cognitively disabled individuals.

It can be appreciated that at least one individual in the space must be monitored, but that any or all others in the space can be monitored as well and subject to the delivery of varying degrees of service and/or protection. The data provided by such a system can be utilized locally, by a live-in or facility-based caregiver, or may be transmitted remotely to family members or other stakeholders concerned about quality of patient care.

In some cases, the data may take the form of alerts, based on a set of conditions that indicate reason for immediate concern. These alerts may be delivered to any individual assigned to intervene in an emergency. This could be a family member, neighbor, or facility staff. Such data is also useful to present a “picture” of living behavior or care delivery over time. Sensor data may be used to confirm delivery of scheduled and/or routine care. Such data may be further utilized to generally indicate the level of socialization of the monitored person—either by their own absence from the monitored space, or through the confirmation of the presence of other individuals within their monitored space.

Furthermore, reliable location information can alert caregivers to potential lapses in the execution of ADLs (Activities of Daily Living) such as eating, bathing, toileting, and mobility within a space. Sudden changes in these behaviors often precede a debilitating health crisis. For example, an elderly patient sick with flu may not be able to make it to the bathroom, or to the kitchen, and suffer serious dehydration before their condition is recognized. An immediate recognition of a change in behavior can therefore increase the likelihood that an informed caregiver may intervene before the situation results in hospitalization and typically, a further decline in independence. Sensor placement in specific areas of activity in a home (e.g., bathroom, kitchen, etc.) can facilitate this assessment. It can be appreciated that in many of the aforementioned cases, the data of interest is not necessarily be a single sensor event, but can be a trend or pattern in the sensor data that indicates a change over time, or a departure from a normal routine.

### BRIEF DESCRIPTION OF THE DRAWINGS

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 illustrates a block diagram of a data-processing apparatus that can be adapted for use in accordance with a preferred embodiment;

FIG. 2(a) illustrates a high-level flow chart of operations depicting logical operational steps for utilizing beacon data to track an unattended person, in accordance with a preferred embodiment;

FIG. 2(b) illustrates a high-level flow chart of operations depicting logical operational steps for utilizing motion sensor data for detecting the presence of a person within a particular area, in accordance with a preferred embodiment;

FIG. 3 illustrates a high-level flow chart of operations depicting logical operation steps for determining the status and monitoring an unattended person, in accordance with a preferred embodiment;

FIG. 4 illustrates an apartment plan or layout of an assisted living facility equipped with a location detection system determining the status and monitoring an unattended person, in accordance with a preferred embodiment;

FIG. 5 illustrates a graph depicting AM routine data of an unattended person in an assisted living facility equipped with a location detection system for determining the status and monitoring the unattended person, in accordance with a preferred embodiment;

FIG. 6 illustrates a graph depicting data that describe the absence of the residents of an assisted living apartment equipped with a location detection system for determining the status and monitoring an unattended person, in accordance with a preferred embodiment;

FIG. 7 illustrates a graph depicting data tracking an individual wearing a beacon at night, in accordance with a preferred embodiment;

FIG. 8 illustrates a graph depicting data tracking an individual not wearing a beacon at night, in accordance with a preferred embodiment;

FIG. 9 illustrates a graph depicting data that describe significant behavior change of a resident of an assisted living apartment equipped with a location detection system for determining the status and monitoring an unattended person, in accordance with a preferred embodiment;

FIG. 10 illustrates a graph depicting spikes in motion profile information with respect to the functionality of a assisted living facility equipped with a location detection system for determining the status and monitoring an unattended person, in accordance with a preferred embodiment;

FIG. 11 illustrates a graph depicting data based on the functionality of a assisted living facility equipped with a location detection system for determining the status and monitoring an unattended person, in accordance with a preferred embodiment; and

FIG. 12 illustrates a block diagram of a system, which can be implemented in accordance with an alternative embodiment.

### DETAILED DESCRIPTION

The particular values and configurations discussed in these non-limiting examples can be varied and are cited merely to illustrate at least one embodiment and are not intended to limit the scope thereof.

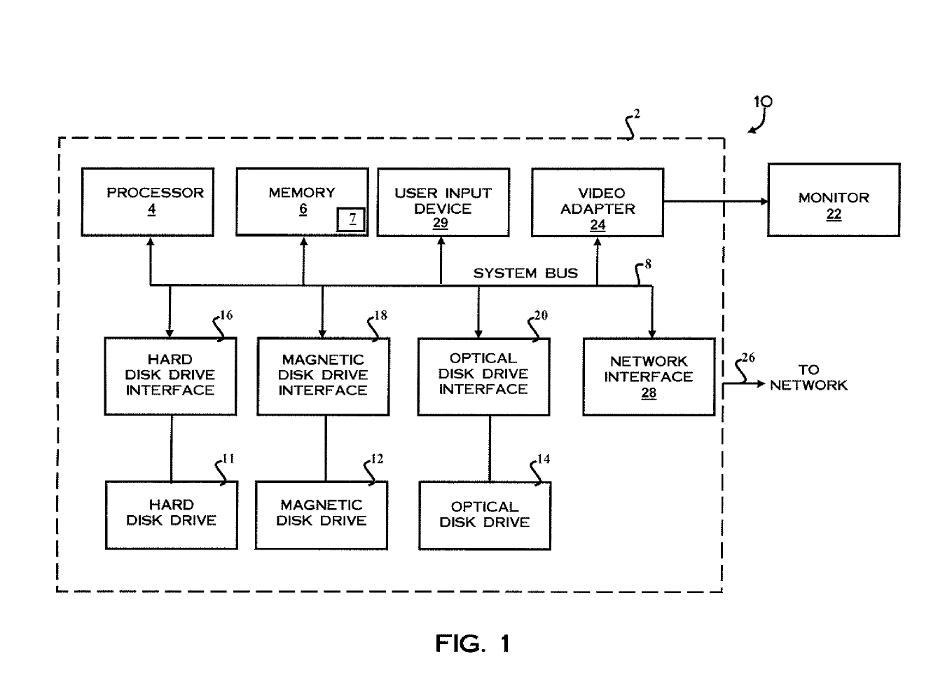

FIG. 1 illustrates a block diagram of a data-processing apparatus 10, which can be utilized to implement a preferred embodiment. Data-processing apparatus 10 can be used to implement a method for distinctively displaying selected building features (e.g., floors) with sufficient details in a three-dimensional building model as described in greater detail herein. Data-processing apparatus 10 can be configured to include a general purpose computing device, such as a computer 2. The computer 2 includes a processing unit 4, a memory 6, and a system bus 8 that operatively couples the various system components to the processing unit 4. One or more processing units 4 operate as either a single central processing unit (CPU) or a parallel processing environment. Data-processing apparatus 10 represents only one of many possible data-processing devices or systems for implementing embodiments. Data-processing apparatus 10 can be provided as a stand-alone personal computer, portable/laptop computer, PDA (personal digital assistant), server, mainframe computer, and so forth.

The data-processing apparatus 10 generally includes one or more data storage devices for storing and reading program and other data. Examples of such data storage devices include a hard disk drive 11 for reading from and writing to a hard disk (not shown), a magnetic disk drive 12 for reading from or writing to a removable magnetic disk (not shown), and an optical disc drive 14 for reading from or writing to a removable optical disc (not shown), such as a CD-ROM or other optical medium. A monitor 22 is connected to the system bus 8 through an adapter 24 or other interface. Additionally, the data-processing apparatus 10 can include other peripheral output devices (not shown), such as speakers and printers. For example, a user input device 29, such as a mouse, keyboard, and so forth, can be connected to system bus 8 in order to permit a user to enter data to and interact with data-processing apparatus 10.

The hard disk drive 11, magnetic disk drive 12, and optical disc drive 14 are connected to the system bus 8 by a hard disk drive interface 16, a magnetic disk drive interface 18, and an optical disc drive interface 20, respectively. These drives and their associated computer-readable media provide nonvolatile storage of computer-readable instructions, data structures, program modules, and other data for use by the data-processing apparatus 10. Note that such computer-readable instructions, data structures, program modules, and other data can be implemented as a module or group of modules, such as, for example, module 7, which can be stored within memory 6.

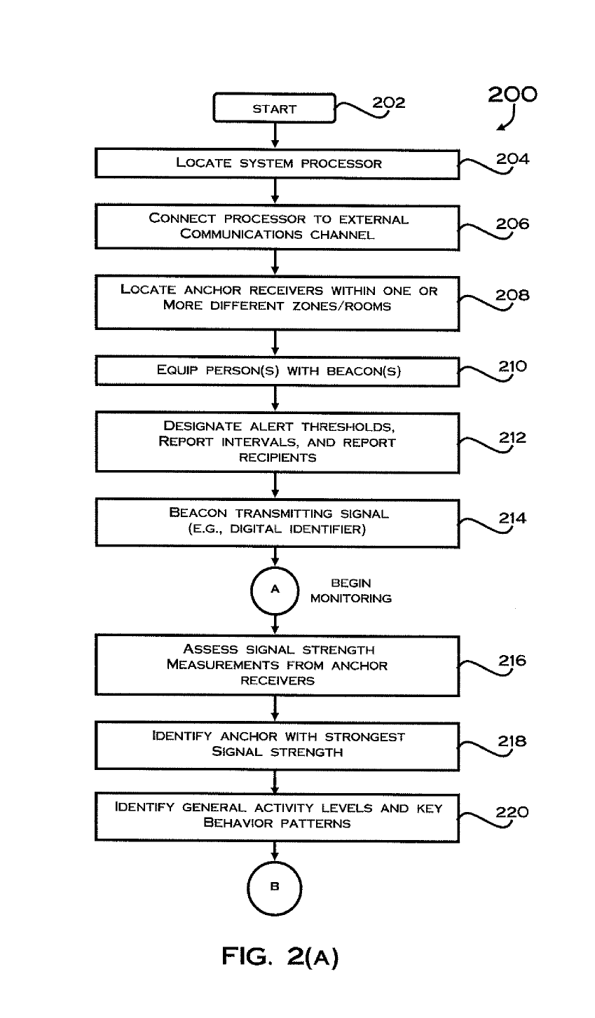

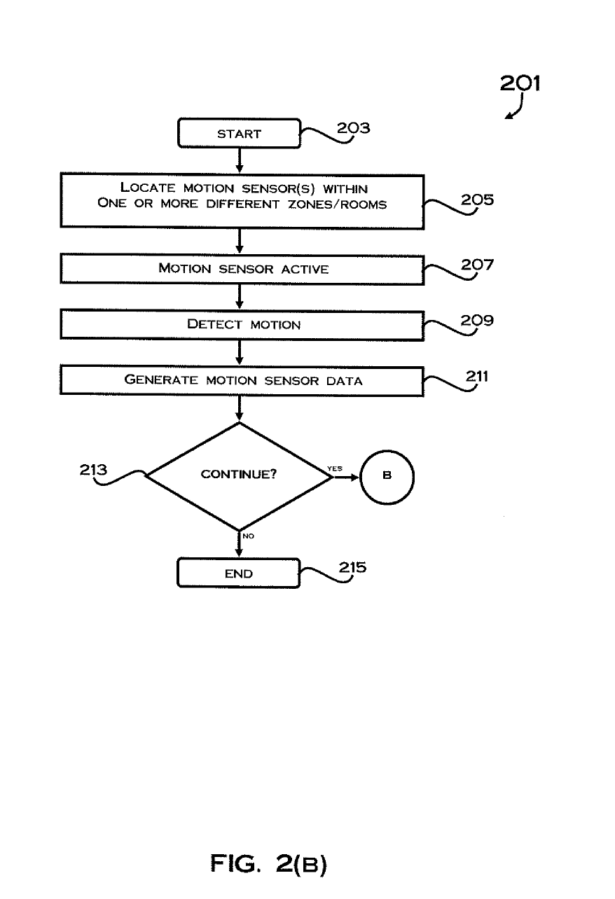

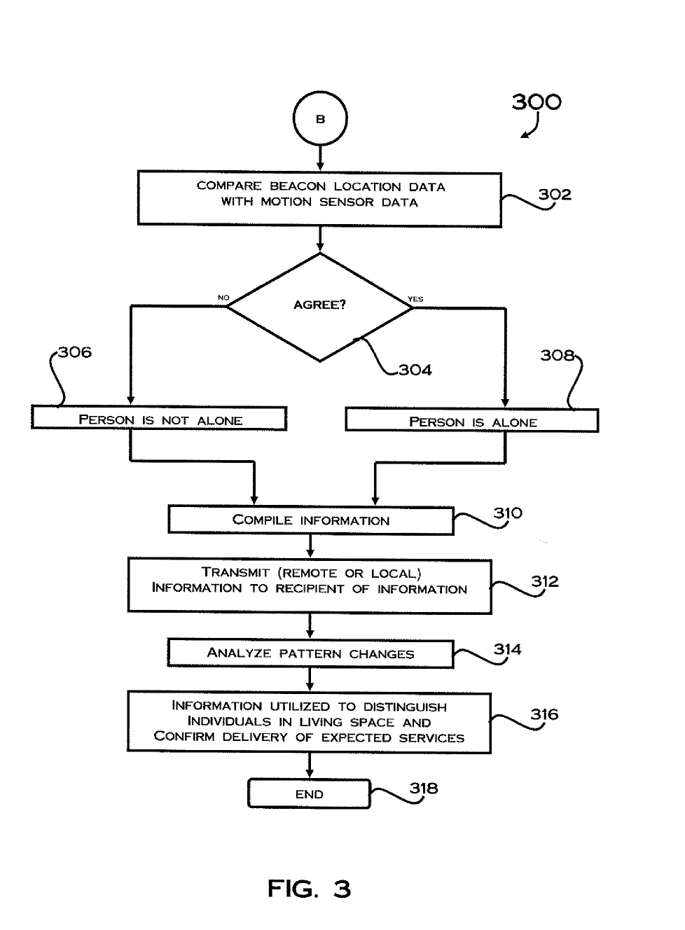

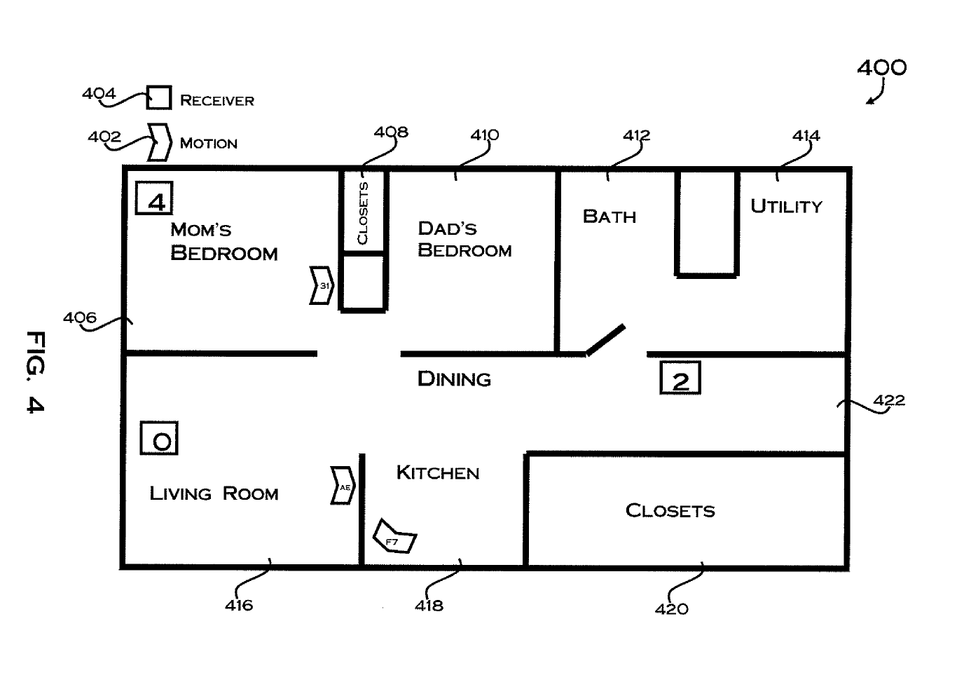

Note that the embodiments disclosed herein can be implemented in the context of a host operating system and one or more module(s) 7. In the computer programming arts, a software module can be typically implemented as a collection of routines and/or data structures that perform particular tasks or implement a particular abstract data type. Module 7 can, for example, implement the methods 200, 300 described and illustrated herein with respect to FIGS. 2 and 3.

Software modules generally comprise instruction media storable within a memory location of a data-processing apparatus and are typically composed of two parts. First, a software module may list the constants, data types, variable, routines and the like that can be accessed by other modules or routines. Second, a software module can be configured as an implementation, which can be private (i.e., accessible perhaps only to the module), and that contains the source code that actually implements the routines or subroutines upon which the module is based. The term module, as utilized herein can therefore refer to software modules or implementations thereof. Such modules can be utilized separately or together to form a program product that can be implemented through signal-bearing media, including transmission media and recordable media.

It is important to note that, although the embodiments are described in the context of a fully functional data-processing apparatus such as data-processing apparatus 10, those skilled in the art will appreciate that the mechanisms of the present invention are capable of being distributed as a program product in a variety of forms, and that the present invention applies equally regardless of the particular type of signal-bearing media utilized to actually carry out the distribution. Examples of signal bearing media include, but are not limited to, recordable-type media such as floppy disks or CD ROMs and transmission-type media such as analogue or digital communications links.

Any type of computer-readable media that can store data that is accessible by a computer, such as magnetic cassettes, flash memory cards, digital versatile discs (DVDs), Bernoulli cartridges, random access memories (RAMs), and read only memories (ROMS) can be used in connection with the embodiments.

A number of program modules can be stored or encoded in a machine readable medium such as the hard disk drive 11, the magnetic disk drive 12, the optical disc drive 14, ROM, RAM, etc or an electrical signal such as an electronic data stream received through a communications channel. These program modules can include an operating system, one or more application programs, other program modules, and program data.

The data-processing apparatus 10 can operate in a networked environment using logical connections to one or more remote computers (not shown). These logical connections are implemented using a communication device coupled to or integral with the data-processing apparatus 10. The data sequence to be analyzed can reside on a remote computer in the networked environment. The remote computer can be another computer, a server, a router, a network PC, a client, or a peer device or other common network node. FIG. 1 depicts the logical connection as a network connection 26 interfacing with the data-processing apparatus 10 through a network interface 28. Such networking environments are commonplace in office networks, enterprise-wide computer networks, intranets, and the Internet, which are all types of networks. It will be appreciated by those skilled in the art that the network connections shown are provided by way of example and that other means of and communications devices for establishing a communications link between the computers can be used.

FIG. 2(a) illustrates a high-level flow chart of operations depicting logical operational steps of a method 200 for utilizing beacon data to track an unattended person, in accordance with a preferred embodiment. FIG. 2(b) illustrates a high-level flow chart of operations depicting logical operational steps of a method 201 utilizing motion sensor data for detecting the presence of a person within a particular area, in accordance with a preferred embodiment. FIG. 3 illustrates a high-level flow chart of operations depicting logical operation steps of a method 300 for determining the status and monitoring an unattended person, in accordance with a preferred embodiment. Note that in FIGS. 2(a), 2(b) and 3, identical or similar parts or elements are generally indicated by identical reference numerals. Methods 200, 201 and 300 can be implemented individually or in combination with one another.

The methodology depicted in FIGS. 2(a), 2(b) and 3 takes into account the fact that location tracking technology can be implemented wherein the location of a mobile beacon is detectable by an array of anchor receivers (also referred to simply as “anchors” or individual as an “anchor”). A beacon located on an object or person can transmit a radio signal that is received by the array of anchors. Since a radio signal attenuates at a known rate over distance, measuring the strength of the signal at the receiver allows the receiver to calculate an estimated distance to the mobile device. Combining the distance measurements from an array of anchor receivers placed in known locations using a triangulation algorithm, allows the location of the beacon to be determined.

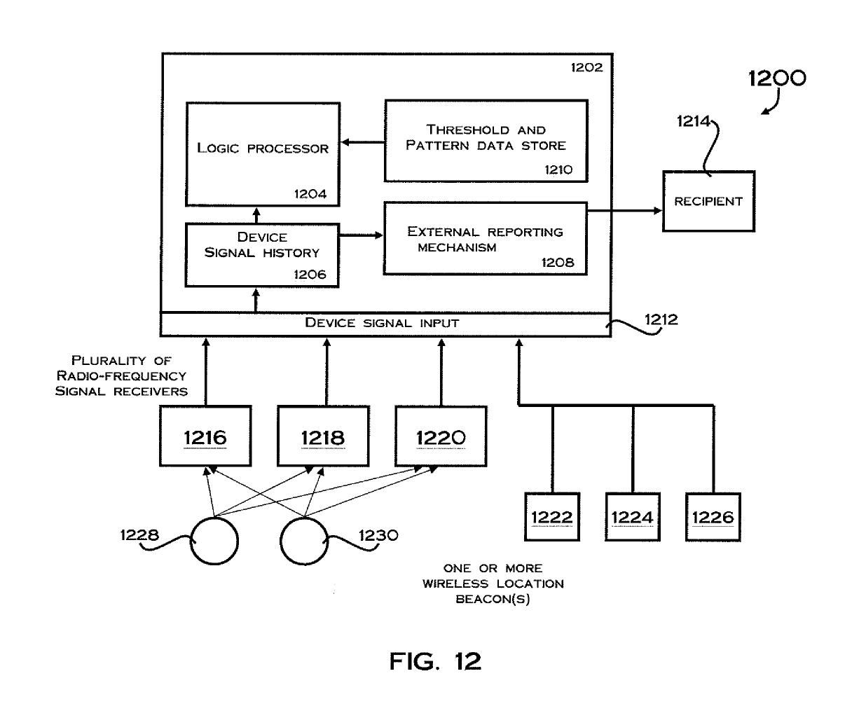

In most home situations, however, it is not cost effective to precisely place each of the anchors. Consequently, the methodology depicted in FIGS. 2(a), 2(b) and 3 provides a solution by locating the anchors only in different rooms or zones of a particular environment, such as a home or assisted living facility. As indicated at block 202 in FIG. 2(a), the process begins. Thereafter, as depicted at block 204, an operation can be processed in which a system processor is located. An example of such a system processor is illustrated herein with respect to FIG. 12 as system processor 1202. Next, as illustrated at block 206, the system processor can be connected to one or more external communications channels. Thereafter, as described at block 208, one or more anchors can be strategically placed in different zones or rooms within the home or assisted living facility. The person being monitored, that is the “unattended person” can be equipped with a radio beacon, as indicated at block 210. Examples of such beacons are depicted in FIG. 12 as beacons 1228 and 1230.

Note that although the methodology discussed herein refers generally to an “unattended person,” the same methodology can be used to track not just one individual but a number of “unattended persons”. That is, more than one unattended individual may be equipped with a unique beacon. The beacon itself can be modified to transmit a digital identifier that uniquely identifies the person wearing the beacon. The use of such a digital identifier allows multiple people to be tracked within a particular area. It is also important to note that the “unattended person(s)” being monitored may be, for example, not only seniors/elderly individuals, but may also be developmentally disabled children or adults. The methodology described herein thus applies to a wide range of individual requiring care, either in a home environment or within an assisted living facility.

Next, as illustrated at block 212, an operation can be processed in which alert thresholds, report intervals, and/or report recipients are designated. Note that an example of a recipient is the recipient 1214 depicted in FIG. 14 herein. Thereafter, as indicated at block 214, the beacon associated with the individual(s) being monitored can transmit a signal. The actual monitoring operations can then take place, as indicated at block A.

The signal strength measurements from each of the anchors can then be assessed as indicated at block 216 and thereafter, as indicated at block 218, the anchor with the strongest signal strength can be utilized to indicate the room or zone in which the unattended person is located. In this manner, the radio beacon associated with the subject person being monitored can be used to track that individual from room to room.

Note that the radio beacon associated with the individual can be, for example, a belt-worn device, a neck pendant, an adhesive body-patch, watch or other form factor carrying a small radio transmitter, depending upon design considerations. Using the radio beacon information alone, it is therefore possible to assess general activity levels, key behavior patterns such as eating in the kitchen, using the bathroom, sleeping in the bedroom, and so forth, as indicated at block 220. Such an assessment can be made by a professional caregiver or family member, depending upon the implemented facility (e.g., a home environment, nursing home, etc.). The information may be transmitted to the recipient, again depending upon design goals and considerations. Such information will not be confounded by the presence of another person within the home or assisted living facility. Following implementation of the operation depicted at block 220, the process continues as indicated at continuation block B.

Method 201 depicted in FIG. 2(b) represents a process that can be implemented in association with the method 200 of FIG. 2(a) for use in monitoring and assessing the activities of an unattended person. As indicated at block 203, the process can begin. Next, as depicted at block 205, one or more sensors can be located within one or more different rooms and/or designated zones of the subject environment (e.g., home, nursing home, etc). Examples of such sensors are depicted in FIG. 12 as motion sensors 1222, 1224, and 1226. The operation described at block 207 indicates that such motion sensors 1222, 1224, and 1226 have been activated and are in operation. Next, as depicted at block 209, the motions sensor(s) 1222, 1224, and 1226 may detect the presence of one or more individuals within a room and/or zone.

The motion sensor will thus detect the presence of any person within the particular room and/or zone in which the motion sensor(s) 1222, 1224, and 1226 are located. The motion sensor data can then be generated at indicated at block 211. Following processing of the operation depicted at block 213, a test can be performed to determine whether to continue with the process or terminate. Assuming, a decision is made to terminate, the process ends as indicated at block 215. If, however, a decision is made to continue, the process continues as indicated at continuation block B. Note that “block B depicted in FIG. 2(a) represents the same logical operational continuation step depicted in FIG. 2(b) and FIG. 3.

Once the methods 200 and 201 have been processed, the methodology depicted of method 300 depicted in FIG. 3 can be implemented. Method 300 combines the information from the radio tracking as indicated by method 200 with the generated motion sensor data resulting from implementation of method 201 in order to determine how the different sources of information agree or differ. Thus, as depicted in FIG. 3, following continuation block B, an operation can be implemented to compare the data resulting from implementation of methods 200 and 201. If the two sources of data agree, it can be assumed that the unattended person is alone as depicted at blocks 222 and 224. Note that inaccuracies that are common in both location tracking approaches will tend to be minimized by combining the two data sources in a manner that provides a more accurate assessment of the actual behavior. When the two sources of information disagree, as indicated in FIG. 3 by blocks 222 and 226, and where motion sensor events occur in rooms other than the room indicated by the location beacon, it can be assumed that there are one or more other persons in the home.

Information concerning the status of the unattended person(s) can then be compiled, as depicted at block 228. The actual transmission of information to the recipient can take place as indicated thereafter at block 232. The transmission may be remote or local and the recipient can be, for example, a professional caregiver or a family member, again depending on the designation by family members or the professional staff of caregivers. Next, as indicated at block 234, an operation can be processed for analyzing the compiled and transmitted information for changes in living patterns over time associated with the unattended person(s). Additionally, such information can be utilized by the professional or family caregiver or other interested parties (e.g., doctors) for distinguishing and confirming the presence of all people in the living space of the unattended person(s) and additionally for confirming the delivery of expecting services such as, for example, bathing, dressing, and feeding. The complied and transmitted information can also be utilized to identify non-human activity such as that caused by pets. Note that in certain circumstances, the “unattended person” (e.g., a patient) may refuse to wear the beacon and may actually remove it if attached to the device. In such cases, all caregivers or other individuals in the area can be “tagged” and the behavior of the patient can be inferred by analyzing the compiled motion data that does not correlate with the caregiver location data. The process can then terminate, as indicated at block 238.

FIG. 4 illustrates an assisted living facility 400 equipped with a location detection system 1200 for determining the status and monitoring an unattended person, in accordance with a preferred embodiment. A detailed view of system 1200 is indicated in FIG. 12. The example apartment plan 400 depicted in FIG. 4 includes sensors 402 and 404 for respectively detecting and receiving motion. Note that sensors 402, 404 are analogous to motion sensors 1222, 1224, and 1226 depicted in FIG. 12. The apartment plan 400 includes a bedroom 406, one or more closets 408, 420 another bedroom 410, a bath 412, a utility room 414, a living room 416, a kitchen 418, and a dining room 422. FIG. 4 thus presents an example scenario that could include two elderly people residing in the assisted living facility 400. Only one individual, however, wore a beacon in the example apartment plan or scenario 400. Additionally, the individuals in question may also eat outside the apartment in a communal dining area. “Kitchen” 418 motion notes passage 422 to bathroom 412. A person wearing a beacon may spend a significant time in his or her bedroom 406. The other individual may spend the majority of his or her time in the living room 416.

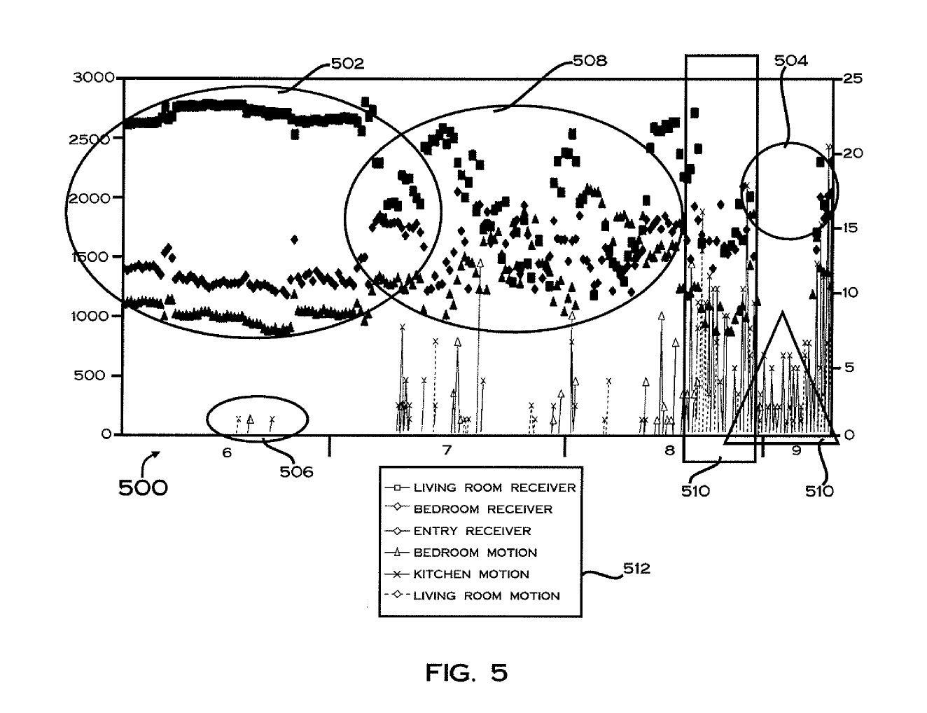

FIG. 5 illustrates a graph 500 depicting data relating to an individual's AM routine in an assisted living facility equipped with a location detection system, in accordance with a preferred embodiment. Graph 500 indicates that an unattended person is resting quietly in his or her bedroom as represented by data 502. Supervisory signals 506 are also depicted in graph 500. These signals provide assurance that the sensors are functioning correctly. Data 508 plotted in graph 500 indicates the movements of an unattended person (e.g., getting up and wandering in and out of the bedroom). Graph 500 indicates that there is sharp increase in motion signals when an aide came to dress the unattended person, as indicated by data 510. Data 514 also indicates that someone is still present in the apartment and this person may be the caregiver. Data 504 depicted in graph 500 also indicates a person leaving the apartment for breakfast 504. Also shown in FIG. 5 in association with graph 500 is a device label 512 that provides tracking data with respect to the living room receiver, bedroom receiver, entry receiver, bedroom motion, kitchen motion and living room motion.

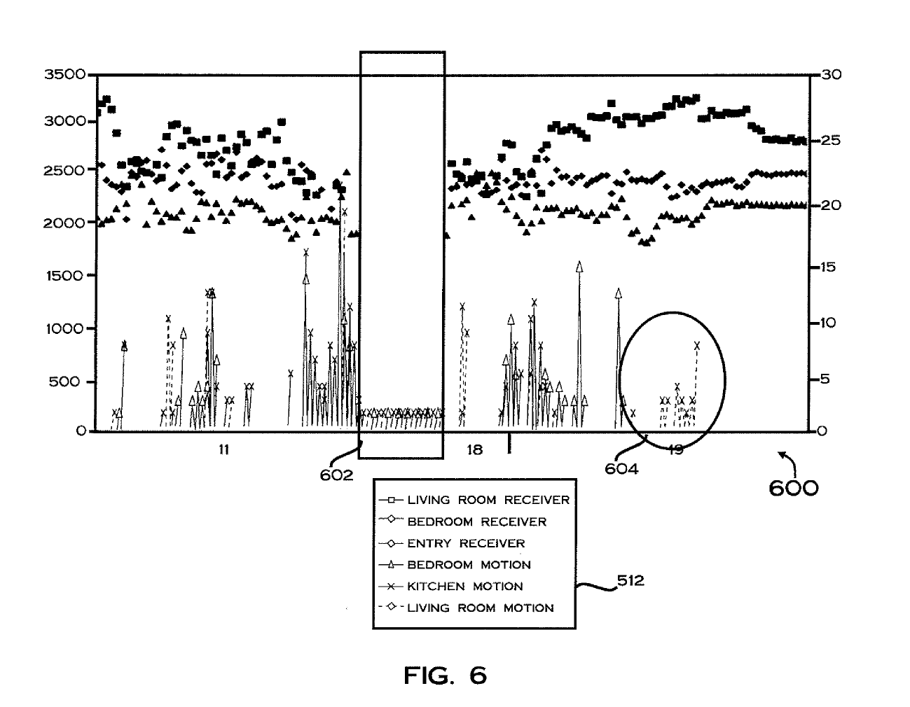

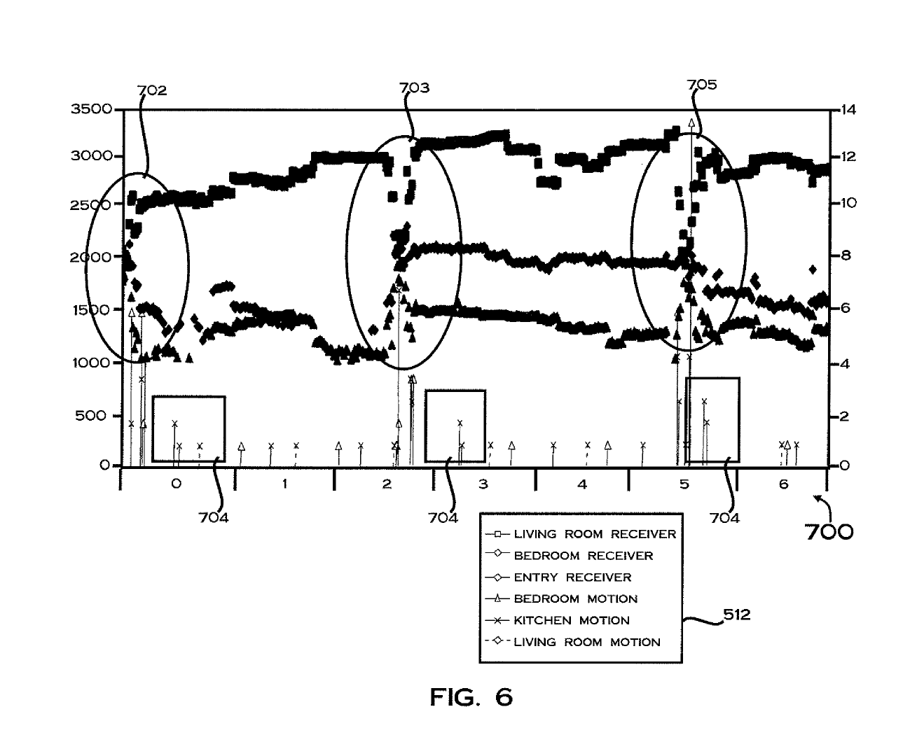

FIG. 6 illustrates a graph 600 of real-world test for determining the status and monitoring an unattended person, in accordance with a preferred embodiment. Graph 600 depicts data indicating the representation of an unattended person who has left his or her apartment to spend day with his or her family. The data depicted in graph 600 indicates the likely presence of a second person in the LR (living room), since the location signal and bedroom motion suggests this individual stayed there. Also shown in Graph 600 is a device label 512 that includes data that tracks the living room receiver, bedroom receiver, entry receiver, bedroom motion, kitchen motion and living room motion. Graph 600 thus relates to data collected according to the scenario or plan 400 of FIG. 4.

FIG. 7 illustrates a graph 700 depicting data related to the wearing of a beacon at night with respect to the functionality of an assisted living facility equipped with a location detection system and real-world test thereof for determining the status and monitoring an unattended person, in accordance with a preferred embodiment. Graph 700 depicts, for example, an unattended person's toileting events 702, 703, 705, and toileting events 704 related to that of a second person. Graph 700 also indicates a device label 512 that provides a legend of data relating to the living room receiver, bedroom receiver, entry receiver, bedroom motion, kitchen motion and living room motion. Graph 700 thus relates to data collected according to the scenario or plan 400 of FIG. 4.

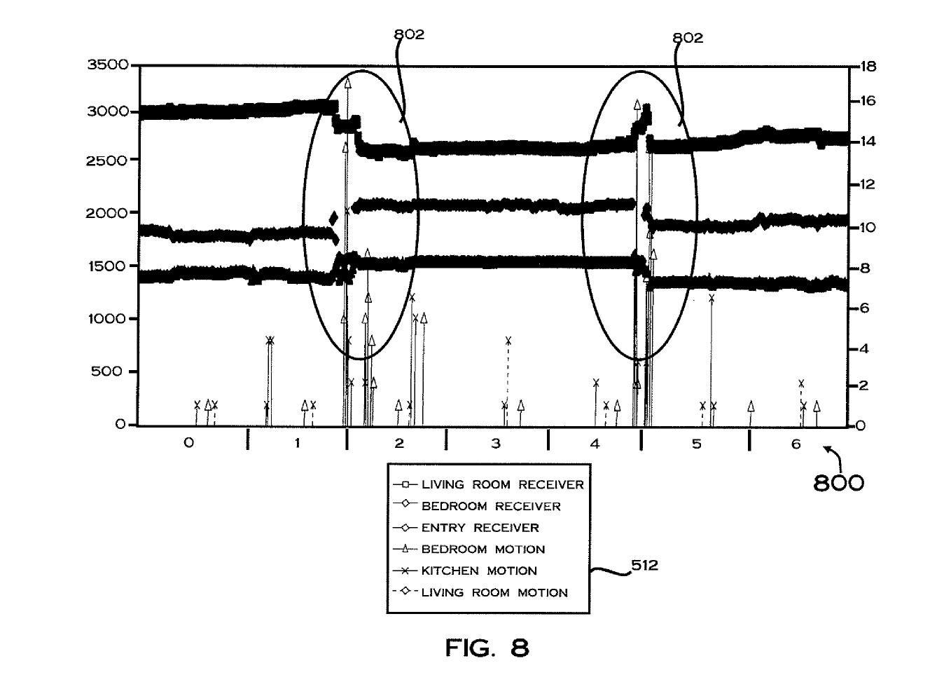

FIG. 8 illustrates a graph 800 depicting data related to not wearing a beacon at night with respect to the functionality of an assisted living facility having a location detection system. Graph 800 tracks real-world test for determining the status and monitoring an unattended person, in accordance with a preferred embodiment. Graph 800 follows the case where, for example, a beacon is left by a person on a dresser all night. Indications of signals, however, are still visible. Graph 800 also indicates a device label 512 that provides a legend of data relating to the living room receiver, bedroom receiver, entry receiver, bedroom motion, kitchen motion and living room motion. Graph 800 thus relates to data collected according to the scenario or plan 400 of FIG. 4.

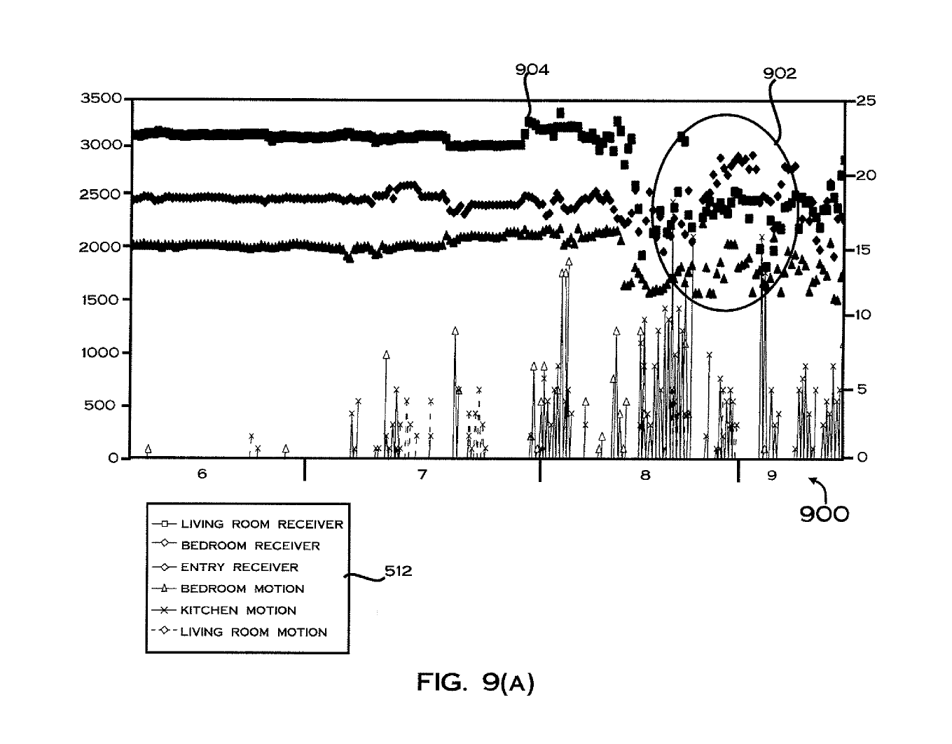

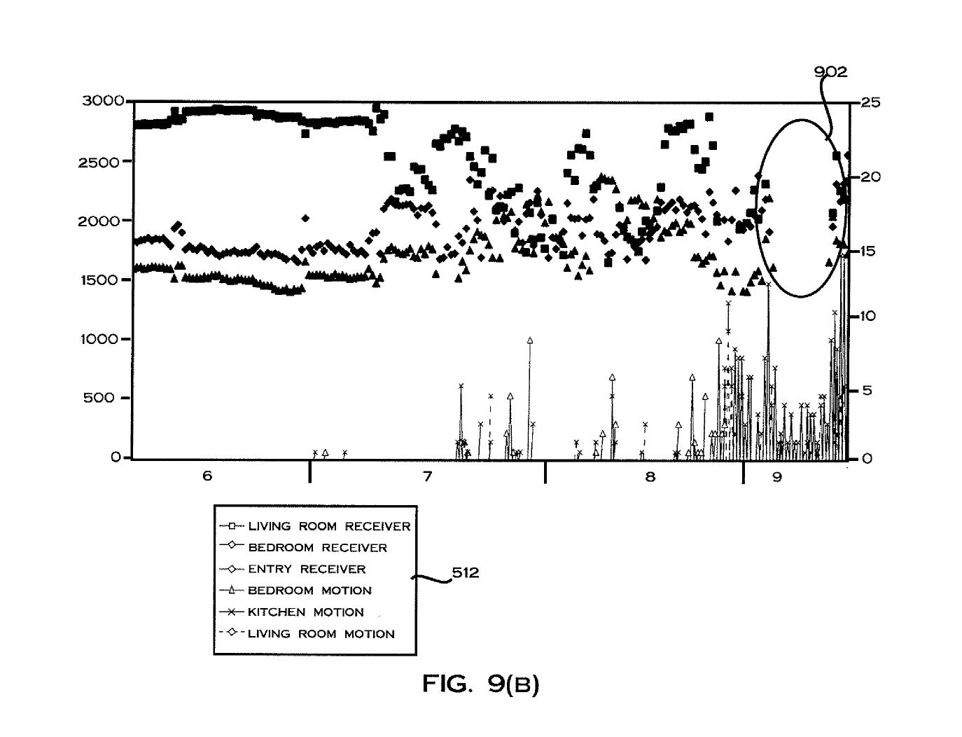

FIG. 9 illustrates a graph 900 depicting data taken over a two-morning time period with respect to the functionality of an assisted living facility having a location detection system, in accordance with a preferred embodiment. Graph 900 tracks real-world test data for determining the status and monitoring an unattended person. As indicated in graph 900, the individual in question did not go out for breakfast in the communal dining area on day 1. Data 902 with respect to the apartment is also tracked in graph 900, along with data 904 indicating that the individual was not wearing a beacon. The device label 512 of graph 900 indicates tracking data with respect to the living room receiver, bedroom receiver, entry receiver, bedroom motion, kitchen motion and living room motion. Graph 900 thus relates to data collected according to the scenario or plan 400 of FIG. 4.

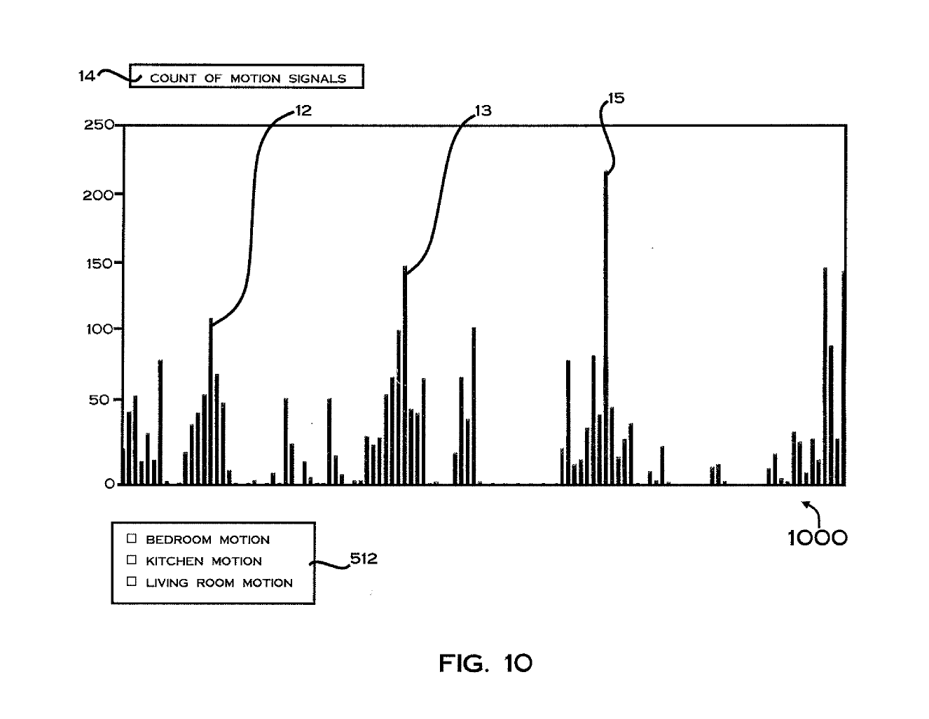

FIG. 10 illustrates a graph 1000 depicting a motion profile with respect to the functionality of an assisted living facility with location detection system information constituting real-world test data for determining the status and monitoring an unattended person, in accordance with a preferred embodiment. Graph 1000 indicates that spikes in motion data correspond to caregiver visits at, for example, 8:30 AM and 12 PM. A count of motion signals 14 is also shown. Spikes 12, 13, 15 are also depicted in FIG. 10. The device label 512 indicates that the graph 1000 relates to data with respect to the living room receiver, bedroom receiver, entry receiver, bedroom motion, kitchen motion and living room motion. Graph 1000 thus relates to data collected according to the scenario or plan 400 of FIG. 4.

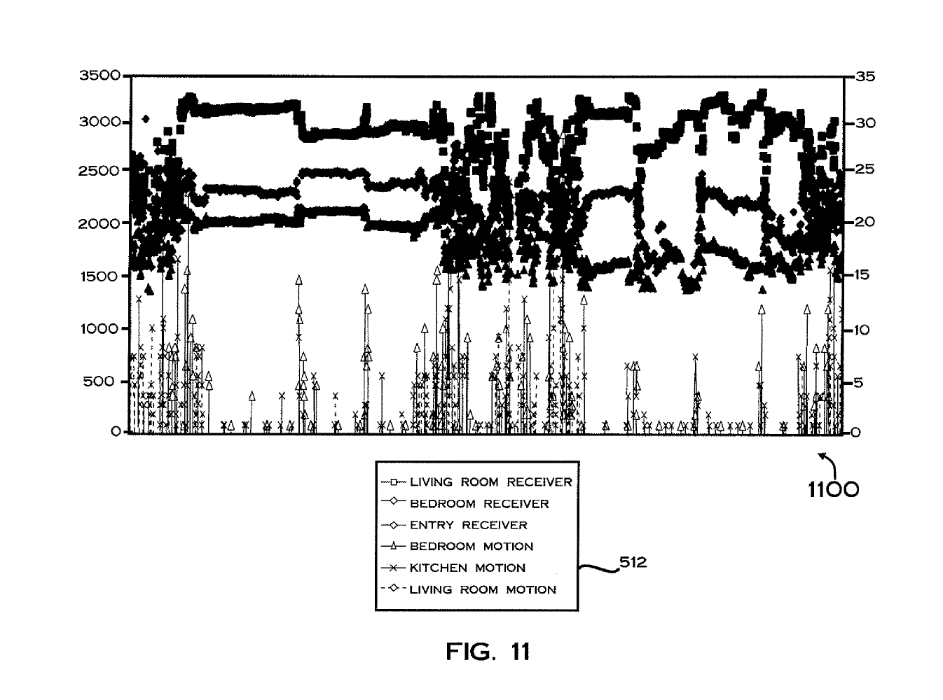

FIG. 11 illustrates a graph 1100 of data associated with the functionality of an assisted living facility equipped with a location detection system for determining the status and monitoring an unattended person, in accordance with a preferred embodiment. The device label 512 depicted in FIG. 11 corresponds to information relating to the living room receiver, bedroom receiver, entry receiver, bedroom motion, kitchen motion and living room motion data. Graph 1100 thus relates to data collected according to the scenario or plan 400 of FIG. 4.

FIG. 12 illustrates a block diagram of a system 1200, which can be implemented in accordance with an alternative embodiment. System 1200 generally includes a system processor 1202 that includes an external reporting mechanism 1208 that can receive data from a logical processor 1204. System processor 1202 also includes a memory 1210 for storing threshold and pattern data provided by the logical operational step described earlier with respect to block 212 of FIG. 2(a). System processor 1202 additionally includes a mechanism 1206 for generating device signal history information, which can be input to the logic processor 1204.

System 1200 further includes one or more wireless location beacons 1228 and 1230, which transmit signals to one or more radio-frequency receivers 1216, 1218, and 1220. A plurality of motion sensors 1222, 1224, and 1226 also generate motion sensor data, which can be provided to a device signal input layer 1212 associated with the system processor 1202. Information generated by the receivers 1216, 1218, and 1220 can also be provided to the device signal input layer of system processor 1202. Finally, data generated by the external reporting mechanism 1208 can be transmitted to the recipient 1214.

Based on the foregoing, it can be appreciated that two separate location techniques can be employed to collect behavioral information about the client/individual and thereby distinguish the presence of other persons in the home or location. One technique involves the use of a radio beacon device carried by the client, and the second technique makes use of an array of motion detection sensors placed in selected rooms or zones of the home. Both systems are capable of determining the room in which the client is located. When the client is alone, both systems will generally show the person in the same room or zone of the home. However, when another person is in the home, the room identified by the two tracking systems will tend to be different. Furthermore, using a radio beacon device, the behavior of the client can be distinguished from the actions of a second person in the home providing more accurate behavioral data about the client. Finally, if the client stops wearing the beacon device, the motion data can still be used as a backup.

Anchors can be placed in different rooms or zones of the home. When the signal strength measurements from each of the anchors are combined, the anchor with the strongest signal strength will be the room or zone in which the person is located. In this manner, the radio beacon can be utilized to track the person from room to room. The radio beacon may be, for example, a belt-worn device, a neck pendant, watch or other form factor carrying a small radio transmitter.

Using the radio beacon information alone, it is thus possible to assess general activity levels, key behavior patterns such as eating in the kitchen, using the bathroom, and sleeping in the bedroom. This information will not be confounded by the presence of another person on the home. Each room or zone will also contain a motion sensor which may or may not be combined with the radio receiver anchor in a single device. The motion sensor will detect the presence of any person in the room.

By combining the information from the radio location tracking with motion sensor data, it will be possible to determine how the different sources of information agree or differ. When the two sources of data agree, it can be assumed that the person is alone. Furthermore, inaccuracies that are common in both location tracking approaches will tend to be minimized by combining the two data sources providing a more accurate assessment of the actual behavior. When the two sources of information disagree where motion sensor events occur in rooms other than the room indicated by the location beacon, it can be assumed that there are one or more other persons in the home.

The confidence of this assessment can be further increased by means of providing a tamper-proof mechanism on the wearable beacon, such that it can be determined whether or not the individual is wearing the device properly. This could take the form of a “smart” latch on a watch, an electricity-conducting element that recognizes contact with skin, or by devising a “patch” whereby the device is adhered to the skin using accepted medically-approved adhesives, and whereby the device can report that it is in contact with a conductive surface (e.g., human skin).

A secondary application of the embodiments described herein is the case of a person with severe cognitive complications (e.g., Alzheimer's disease, autism, etc.). In such a situation, the embodiments described herein offer the ability to track other people in the environment by means of wearable devices, such that their movements can be distinguished from those of the untracked patient in the environment. In this case, the wearable beacon can be utilized to automatically disarm the system, or prevent alerts from being raised due to the movements of the caregiver, while the system would still appropriately alert based on movements of the patient, or monitored individual. In this case, the wearable beacon may also function as a communication device, thereby allowing direct communication between the monitoring system and the monitoring caregiver, who is wearing the device.

It will be appreciated that variations of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A method for determining the status an individual, comprising:
associating a radio beacon device with an individual;
collecting behavioral information about said individual by tracking a location of said individual within a particular area utilizing said radio beacon device; and
distinguishing a presence of other persons from said individual within said particular area utilizing said behavioral information about said individual, thereby determining a status of said individual and distinguishing the presence of other persons within said particular area.

2. The method of claim 1 wherein said behavioral information comprises a plurality of activity levels associated with said individual.

3. The method of claim 1 further comprising placing an array of motion detection sensors within at least one selected location of said particular area, wherein said array of motion detection sensors generates motion sensor data.

4. The method of claim 3 utilizing said motion sensor data generated by said array of motion detection sensors and said behavioral information associated with said individual to determine if said motion sensor data and said behavioral information agree or differ.

5. The method of claim 4 wherein if said motion sensor data and said behavioral information do agree with each other, an assumption is generated indicating that said individual is alone within said particular area.

6. The method of claim 4 wherein if said motion sensor data and said behavioral information do not agree with each other, an assumption is generated indicating that more than one individual is present within said particular area.

7. The method of claim 1 further comprising applying a triangulation algorithm for determining a location of said radio beacon device located with said individual.

8. A system for determining the status an individual, comprising:
a radio beacon device associated with an individual;
a data-processing apparatus;
a module executed by said data-processing apparatus, said module and said data-processing apparatus being operable in combination with one another to:
collect behavioral information about said individual by tracking a location of said individual within a particular area utilizing said radio beacon device; and
distinguish a presence of other persons from said individual within said particular area utilizing said behavioral information about said individual, thereby determining a status of said individual and distinguishing the presence of other persons within said particular area.

9. The system of claim 8 wherein said behavioral information comprises a plurality of activity levels associated with said individual.

10. The system of claim 8 further comprising an array of motion detection sensors located within at least one selected location of said particular area, wherein said array of motion detection sensors generates motion sensor data.

11. The system of claim 10 wherein said module and said data-processing apparatus are operable in combination with one another to utilize said motion sensor data generated by said array of motion detection sensors and said behavioral information associated with said individual to determine if said motion sensor data and said behavioral information agree or differ.

12. The system of claim 11 wherein if said motion sensor data and said behavioral information agree with each other, an assumption is generated by said module and said data-processing apparatus operable in combination with one another indicating that said individual is alone within said particular area.

13. The system of claim 11 wherein if said motion sensor data and said behavioral information do not agree with each other, an assumption is generated by said module and said data-processing apparatus operable in combination with one another indicating that more than one individual is present within said particular area.

14. The system of claim 1 wherein said module and said data-processing apparatus are operable in combination with one another to apply a triangulation algorithm for determining a location of said radio beacon device located with said individual.

15. A program product for determining the status an individual, comprising:
instruction media residing in a computer for associating a radio beacon device with an individual;
instruction media residing in a computer for collecting behavioral information about said individual by tracking a location of said individual within a particular area utilizing said radio beacon device; and
instruction media residing in a computer for distinguishing a presence of other persons from said individual within said particular area utilizing said behavioral information about said individual, thereby determining a status of said individual and distinguishing the presence of other persons within said particular area.

16. The program product of claim 15 wherein said behavioral information comprises a plurality of activity levels associated with said individual.

17. The program product of claim 15 further comprising an array of motion detection sensors located within at least one selected location of said particular area, wherein said array of motion detection sensors generates motion sensor data.

18. The program product of claim 17 further comprising instruction media residing in a computer for utilizing said motion sensor data generated by said array of motion detection sensors and said behavioral information associated with said individual to determine if said motion sensor data and said behavioral information agree or differ.

19. The program product of claim 18 wherein:
if said motion sensor data and said behavioral information agree with each other, an assumption is generated indicating that said individual is alone within said particular area; and
if said motion sensor data and said behavioral information do not agree with each other, an assumption is generated indicating that more than one individual is present within said particular area.

20. The method of claim 1 further comprising instruction media residing in a computer for applying a triangulation algorithm for determining a location of said radio beacon device located with said individual.
